SEQUENCE LISTING (1 sequence)

SEQ ID NO: 1 - protein, 21 aa, Artificial sequence
  PEPTIDE 35..55: Sequence of myelin oligodendrocyte glycoprotein peptide
MEVGWYRSPFSRVVHLYRNGK

(19) United States
(12) Patent Application Publication
(10) Pub. No.: US 2017/0260283 A1
(43) Pub. Date: Sep. 14, 2017

(54) Oral Administration of an Anti-CD20 Antibody for Treatment of Autoimmune Disease

(71) Applicant: Staley A. Brod, Weston, MA (US)

(72) Inventor: Staley A. Brod, Weston, MA (US)

(21) Appl. No.: 15/063,822

(22) Filed: Mar. 8, 2016

(51) Int. Cl. C07K 16/28; A61K 9/00; A61K 45/06; A61K 39/395

(52) U.S. Cl. CPC C07K 16/2887; A61K 39/3955; A61K 9/0053; A61K 45/06; A61K 2039/505

(57) ABSTRACT

The present invention provides a method for treating or delaying the onset of an autoimmune condition in a human subject. An effective oral dose of an anti-CD20 antibody is administered to the subject. Oral administration of such antibodies as rituximab, ocrelizumab, ofatumumab, obinutuzumab, tositumomab, or ibritumomab are useful in a method of decreasing innate inflammatory cytokines, such as and TNF-a, IFN-g, IL-17, and IL-12.

### BACKGROUND OF THE INVENTION

Field of the Invention

The present invention relates generally to the fields of autoimmune diseases. More specifically, the present invention relates to uses of ingested (orally administered) anti-CD20 antibody in the treatment of autoimmune diseases.

Description of the Related Art

The following abbreviations may be used herein: ACTH: Adrenocorticotropin hormone, a-MSH: alpha-melanocyte stimulating hormone, EAE: experimental autoimmune encephalomyelitis, DMARD: disease modifying anti-rheumatic drugs, GALT: gut associated lymphoid tissue, RTX: rituximab, SIRS: soluble immune response suppressor, SST: somatostatin, T_{reg}: T regulatory cell.

EAE is a T cell mediated inflammatory autoimmune process of the CNS that resembles the human demyelinating disease multiple sclerosis (MS) (1) and provides a useful animal model for the evaluation of potential therapies for cellular mediated autoimmune diseases (2-4). Ingested proteins such as type I IFN (5) SIRS peptide 1-21 (6) a-MSH (7) ACTH (8) and SST (6) inhibit attacks and inflammation in acute EAE (9-10). B cells may also play an important part in EAE (11-12).

In 1997, a chimeric anti-CD20 monoclonal antibody (mAb) (Rituxan) was approved for the treatment of B-cell lymphoma with human IgG₁ constant domains (13). Crosslinking of CD20 molecules by Rituxan induces therapeutic B-cell depletion (14). Rituxan has a long half-life and low immunogenicity, and it mediates effector function (15).

B cell-driven pathogenesis in T cell-mediated autoimmune disease is a major mechanism in EAE and MS (11). B cell depletion in MS using a mAb to CD20 (rituximab) has shown promising results (16) and leads to amelioration of disease irrespective of autoantibody ablation. Efficacy of Rituxan treatment has been reported in nonmalignant autoimmune diseases such as rheumatoid arthritis (14). Additionally, recent clinical trials have established B cell depletion by the anti-CD20 chimeric antibody Rituximab as a beneficial therapy for patients with relapsing-remitting multiple sclerosis (MS) (17-19).

B cells can have pleitropic effect on EAE initiation and recovery. Recovery can be dependent on the presence of autoantigen-reactive B cells in response to autoantigen (20) B cells regulate the extent of EAE clinical disease and their absence exacerbated disease (16). In MOG p35-55 as opposed to recombinant myelin oligodendrocyte glycoprotein (rMOG)-induced EAE, anti-CD20 treatment exacerbated EAE and did not impede development of Th1 or Th17 cells (20).

In contrast, B-lymphocyte deficient rats failed to develop clinical or histological evidence of EAE when sensitized with either whole spinal cord or purified BP (21). The development of spontaneous EAE in TCR Tg MOG 92-106 depends on the presence of an intact B cell compartment (22). B cell depletion during EAE disease progression (day 14) dramatically suppressed symptoms compared to B cell depletion during EAE induction (23). Depletion of peripheral B cells strongly reduced EAE severity and IL-17 production (24).

Therefore, the prior art is deficient in the use of oral, ingested anti-CD20 antibody in the treatment of autoimmune diseases such as multiple sclerosis. The present invention fulfills this long-standing need and desire in the art.

### SUMMARY OF THE INVENTION

The present invention is directed to a method for treating or delaying the onset of an autoimmune condition in a human subject. The method comprises orally administering to the subject an effective dose of an anti-CD20 antibody.

The present invention also is directed to a method of decreasing innate inflammatory cytokines TNF-α, IFN-γ, IL-17, IL-12. The method comprises orally administering to the subject an effective dose of an anti-CD20 antibody.

Other and further aspects, features, and advantages of the present invention will be apparent from the following description of the presently preferred embodiments of the invention. These embodiments are given for the purpose of disclosure.

### BRIEF DESCRIPTION OF DRAWINGS

The following drawings are part of the present specification and are included to further demonstrate certain aspects of the present invention. The invention may be better understood by reference to the drawings in combination with the detailed description of specific embodiments presented herein.

FIG. 1 shows that ingested Rituxan inhibits EAE attacks. B6 mice (n=8/group) were immunized with MOG peptide 35-55 and were gavaged with 0.1 ml of 1 μg or 10 μg IgG isotype control or 1 mg or 10 mg Rituxan. Both 1 and 10 mg ingested Rituxan significantly inhibits EAE progression compared to control (p<0.001, ANOVA, day 17-37, group score ±SEM). The figure shows combined results from 3 separate experiments (total n=24/group).

FIG. 2 shows that adoptively transferred donor cells from Rituxan fed immunized mice protect against active EAE. Thirty two days after inoculation and peak score of attack, spleens from 1 mg IgG control and 1 mg Rituxan fed mice were isolated and re-stimulated with MOG peptide 35-55 and adoptively transferred. A third group of control spleen cells were re-stimulated with MOG peptide 35-55 and 50 mg/ml Rituxan in vitro. Recipients of IgG control fed donor cells increased their group disease severity. The in vitro control group 50 mg/ml IgG isotype control is not different from fed IgG control. In contrast, recipients of Rituxan fed donor cells and donor cells incubated with Rituxan in vitro decreased their group score significantly compared to recipients of saline control cells (p<0.005, days 17-32, ANOVA, group score ±SEM). There was a significantly better score with 1 mg RTX fed group vs the 50 mg/ml RTX in vitro group (p<0.05). This experiment shows a combination of 3 separate experiments (total n=24/group).

FIG. 3 shows that adoptively transferred donor CD4⁺ T cells from Rituxan fed immunized mice protect against active EAE. Thirty one days after inoculation and after peak score of attack, spleens from IgG control and 1 mg Rituxan fed mice were isolated and re-stimulated with MOG peptide 35-55, CD4⁺ T cells isolated and adoptively transferred. Recipients of IgG control fed CD4⁺ T donor cells increased their group disease severity. In contrast, recipients of Rituxan fed CD4⁺ T cells donor cells decreased their group score significantly compared to recipients of saline control cells (p<0.005, days 17-31, ANOVA, group score ±SEM). This experiment shows a combination of 3 separate experiments (total n=12/group).

FIG. 4 shows that ingested Rituxan decreases pro-inflammatory Th1-like and IL-17 cytokines in the spleens of actively immunized mice. Lymphocytes isolated from spleen cells from IgG control fed mice or Rituxan fed mice were stimulated with MOG peptide 35-55 and measured using an inflammatory cytokine antibody array as described in methods. Splenic lymphocytes showed decreased levels of TNF-α (p<0.01), Th1-like cytokine IFN-γ (p<0.001), IL-12 (p<0.01) and IL-17 (p<0.01). This experiment shows a combination of 4 separate experiments (total n=16/group). Results are expressed as pg/ml ±SEM.

FIG. 5 shows recipients of donor cells from Rituxan fed mice show decreases pro-inflammatory Th1-like and IL-17 cytokines. Lymphocytes isolated from spinal cords from recipients of IgG control fed or Rituxan fed donor cells were stimulated with MOG peptide 35-55 and measured using an inflammatory cytokine antibody array as described below. CNS lymphocytes showed decreased levels of TNF-α (p<0.01), Th1-like cytokine IFN-γ (p<0.01), IL-12 (p<0.03) and IL-17 (p<0.03) in Rituxan dosed vs IgG control dosed mice. This experiment shows a combination of 4 separate experiments (total n=16/group). Results are expressed as pg/ml ±SEM.

FIG. 6 shows that recipients of donor cells from Rituxan fed mice show decrease pro-inflammatory Th1-like and IL-17 cytokines. Lymphocytes isolated from spleens from recipients of IgG control fed or Rituxan fed donor cells were stimulated with MOG peptide 35-55 and measured using an inflammatory cytokine antibody array as described below. Splenic lymphocytes showed decreased of TNF-α (p<0.001), Th1-like cytokine IFN-g (p<0.01), IL-12 (p<0.01) and IL-17 (p<0.01) in Rituxan dosed vs IgG control dosed mice. This experiment shows a combination of 4 separate experiments (total n=16/group). Results are expressed as pg/ml ±SEM.

FIG. 7 shows that recipients of donor cells from Rituxan fed mice show decrease pro-inflammatory Th1-like and IL-17 cytokines. Lymphocytes isolated from spinal cords from recipients of IgG control fed or Rituxan fed donor cells were stimulated with MOG peptide 35-55 and measured using an inflammatory cytokine antibody array as described below. CNS lymphocytes showed decreased of TNF-α (p<0.01), Th1-like cytokine IFN-γ (p<0.01), IL-12 (p<0.01) and IL-17 (p<0.01) in Rituxan dosed vs IgG control dosed mice. This experiment shows a combination of 4 separate experiments (total n=16/group). Results are expressed as pg/ml ±SEM.

FIG. 8 shows that recipients of Rituxan fed spleen CD4⁺ T cells donor cells from Rituxan fed mice show decreases pro-inflammatory Th1-like and IL-17 cytokines. The cytokine profiles of MOG re-stimulated spleen lymphocytes were examined in recipients of IgG fed CD4⁺ T cells vs Rituxan fed CD4⁺ T cells donor cells (from FIG. 3). Recipient splenic lymphocytes Th1-like cytokines TNF-α, IL-12/IFN-γ and IL-17 after CD4⁺ T cells transfer in Rituxan fed groups compared to the IgG fed group. Splenic lymphocytes showed decreased of TNF-α (p<0.001), Th1-like cytokine IFN-γ (p<0.001), IL-12 (p<0.01) and IL-17 (p<0.001) in Rituxan dosed vs IgG control dosed mice. Results are expressed as pg/ml ±SEM.

### DETAILED DESCRIPTION OF THE INVENTION

As used herein, the term “a” or “an” when used in conjunction with the term “comprising” in the claims and/or the specification may mean “one,” but it is also consistent with the meaning of “one or more,” “at least one,” and “one or more than one.”

As used herein “another” or “other” may mean at least a second or more of the same or different claim element or components thereof. Similarly, the word “or” is intended to include “and” unless the context clearly indicates otherwise. “Comprise” means “include.”

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the term “subject” refers to any human or non-human animal that is the recipient of an anti-CD20 antibody as described herein.

Treatment of chronic autoimmune disease is challenging even with the advent of new therapeutic techniques. Typical therapies involve the administration of immunosuppressive agents such as steroids. Though steroids are typically not highly effective, they are well tolerated for long term use and many may be administered orally. A non-invasive method for administration, such as oral administration, is highly preferred in cases of chronic diseases such as multiple sclerosis.

The new methods disclosed herein address one of the greatest obstacles to treating chronic disease such autoimmune disease, that is long term tolerance of the therapeutic regimen. Such tolerance takes into account not only biological tolerance, but also tolerance in patients undergoing therapy. Injectable therapeutics are far from ideal for the treatment of chronic disease. Consent injection can result in lasting damage to the tissues around the injection site and is painful and inconvenient for patients. Additionally, injection of any substance into the body increases the risk for infection by bacteria or viruses that may be present in the therapeutic formulations or on the injection apparatus itself. The instant invention enables methods for oral administration of potent immunomodulatory polypeptides. Surprisingly, these polypeptides remain highly active in an oral formulation and are effective for treating autoimmune disease. These new oral therapeutic polypeptides are particularly well adapted for prolonged administration that is often required for the treatment of chronic disease.

Anti-CD20 antibody compositions according to the instant invention may also be used in conjunction with other therapies that are used for the treatment of inflammation and/or autoimmune diseases. Such secondary therapies can include small molecule drugs as well as therapeutic nucleic acids or polypeptides. Anti-inflammatory agents, for example, are agents that decrease signs and symptoms of inflammation. A wide variety of anti-inflammatory agents are known to one of skill in the art. Most commonly used are the nonsteroidal anti-inflammatory agents (NSAIDs) which work by inhibiting the production of prostaglandins. Non-limiting examples include, ibuprofen, ketoprofen, piroxicam, naproxen, naproxen sodium, sulindac, aspirin, choline subsalicylate, diflunisal, oxaprozin, diclofenac sodium delayed release, diclofenac potassium immediate release, etodolac, ketorolac, fenoprofen, flurbiprofen, indomethacin, fenamates, meclofenamate, mefenamic acid, nabumetone, oxicam, piroxicam, salsalate, tolmetin, and magnesium salicylate. Another group of anti-inflammatory agents comprise steroid based potent anti-inflammatory agents, for example, the corticosteroids which are exemplified by dexamethason, hydrocortisone, methylprednisolone, prednisone, and triamcinolone as non-limiting examples. Several of these anti-inflammatory agents are available under well known brand names, for example, the NSAIDs comprising ibuprofen include Advil, Motrin IB, Nuprin; NSAIDs comprising acetaminophens include Tylenol; NSAIDs comprising naproxen include Aleve.

As discussed supra, certain known immunomodulatory polypeptides may also be used in accordance with the invention. Such polypeptides include, but are not limited to, SIRS, interferon-alpha and interferon-tau.

Pharmaceutical compositions of the present invention comprise an effective amount of anti-CD20 antibody and optionally at least one additional agent dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains an anti-CD20 antibody or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, gels (e.g., gelatin), dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

A pharmaceutical composition of the present invention comprising an anti-CD20 antibody may also comprise different types of carriers depending on whether it is to be administered in solid, liquid or aerosol form, and whether it need to be sterile. The present invention can be administered intranasally, intravitreally, intravaginally, intrarectally, topically, mucosally, intraocularally, orally, topically, locally, via inhalation (e.g. aerosol inhalation), via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of an anti-CD20 antibody composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof. In the case of proteinaceous compositions of the invention, it may also be preferable that the action of proteases be inhibited during storage of such anti-CD20 antibody compositions. This can be accomplished by the additional of protease inhibitors and/or the storage of the compositions at low temperature prior to administration.

In embodiments where compositions according to the invention are provided in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In certain embodiments, an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof an excipient, such as, for example, dicalcium phosphate, mannitol, lactose, starch, magnesium stearate, sodium saccharine, cellulose, magnesium carbonate or combinations thereof a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Methods of the invention will generally be used in an amount of an anti-CD20 antibody effective to achieve the intended purpose. For use to treat or prevent a disease condition, antibodies such as rituximab, ocrelizumab, ofatumubmab, obinutuzumab, tositumomab, or ibritumomab, or pharmaceutical compositions thereof, are administered in a therapeutically effective amount. A therapeutically effective amount is an amount effective to ameliorate or prevent the symptoms, or prolong the survival of, the patient being treated. Determination of a therapeutically effective amount is well within the capabilities of those skilled in the art, especially in light of the detailed disclosure provided herein. The amount of molecules administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs. In the case of autoimmune disorders, the drugs that may be used in combination with an anti-CD20 antibody include, but are not limited to, steroid and non-steroid anti-inflammatory agents.

Methods for estimating dose conversions between animal models and humans have been developed. In general these algorithms have been used to extrapolate an animal dose to a dose that would be tolerated by a human. For example, method for dose conversions were disclosed by Freireich et al. (26). The conversion methods taught by Freireich calculate equivalent doses between species using surface area (m²) rather than mass (kg), a method that correlates much more closely to actual data than body mass conversions. Specifically, Freireich teaches how to use an animal 10% lethal dosage (LD₁₀) value to estimate the maximum tolerated doses in a human. Freireich also discussed method for converting a dose in mg/kg to a dose in mg/m² by using the “km” conversion factor for the given animal.

More recent studies regarding species dose scaling have further elaborated upon the methods of Freireich. These newer studies have reduced error associated with conversion between species to determine human tolerable doses. For example, Watanabe et al. (27) describes that a conversion of doses between species using body surface area may not be the most accurate method per se for predicting a human equivalent dosage. Nonetheless, the scaling factors set forth by Watanabe yield results that are within the margin of error of the older Freireich conversions. Currently accepted methods for determining a proper starting dose in humans expand upon the methods set forth by Freireich. For example, Mahmood et al. (28) provides a discussion regarding the choice of a proper starting dose in humans given dose studies in animals.

As described in detail below, the present invention is directed to a method for treating or delaying the onset of an autoimmune condition in a human subject comprising orally administering to the subject an effective dose of an anti-CD20 antibody. In one aspect of this method, the anti-CD20 antibody is administered in a liquid form. In one aspect of this method, the anti-CD20 antibody is administered in a solid form. Representative examples of anti-CD20 antibodies include but are not limited to rituximab, ocrelizumab, ofatumubmab, obinutuzumab, tositumomab, veltuzumab, GA101, TRU-015, PRO131921, or ibritumomab. Representative examples of condition include but are not limited to rheumatoid arthritis, psoriasis, type 1 diabetes, systemic lupus erythematosus, transplant rejection, autoimmune thyroid disease (Hashimoto's disease), sarcoidosis, scleroderma, granulomatous vasculitis, Crohn's disease, ulcerative colitis, Sjogren's disease, ankylosing spondylitis, polymyositis dermatomyositis, polyarteritis nodosa, immunologically mediated blistering skin diseases, Behcet's syndrome, multiple sclerosis, systemic sclerosis, Goodpasture's disease or immune mediated glomerulonephritis. A person having ordinary skill in this are would be able to prepare satisfactory composition of an anti-CD20 antibody and readily determine appropriate dosages for the condition to be treated. For example, an anti-CD20 antibody may be administered in a dose from about from about 0.001 mg to about 50 mg.

In one preferred embodiment, an anti-CD20 antibody is administered in a dose from about 3-30 mg. Generally, the anti-CD20 antibody administration decreases levels of IL-12, IL-17, TNF-α and IFN-γ. In a preferred embodiment, the anti-CD20 antibody may be administered in combination with a drug such as an anti-inflammatory agent, a SIRS peptide, α-MSH, ACTH and SST. Representative examples of anti-CD20 antibodies include but are not limited to rituximab, ocrelizumab, ofatumubmab, obinutuzumab, tositumomab, veltuzumab, GA101, TRU-015, PRO131921, or ibritumomab.

In another embodiment, the present invention also provides a method of decreasing innate inflammatory cytokines TNF-α, and IFN-γ, IL-17, IL-12 in a human subject comprising orally administering to the subject an effective dose of an anti-CD20 antibody. In one aspect of this method, the an anti-CD20 antibody is administered in a liquid or a dry form. In one aspect of this method, the anti-CD20 antibody is administered in a solid form. Representative examples of condition include but are not limited to rheumatoid arthritis, psoriasis, type 1 diabetes, systemic lupus erythematosus, transplant rejection, autoimmune thyroid disease (Hashimoto's disease), sarcoidosis, scleroderma, granulomatous vasculitis, Crohn's disease, ulcerative colitis, Sjogren's disease, ankylosing spondylitis, polymyositis dermatomyositis, polyarteritis nodosa, immunologically mediated blistering skin diseases, Behcet's syndrome, multiple sclerosis, systemic sclerosis, Goodpasture's disease or immune mediated glomerulonephritis. A person having ordinary skill in this are would be able to prepare satisfactory composition of an anti-CD20 antibody and readily determine appropriate dosages for the condition to be treated. For example, the anti-CD20 antibody may be administered in a dose from about from about 0.001 mg to about 50 mg. Representative examples of anti-CD20 antibodies include but are not limited to rituximab, ocrelizumab, ofatumubmab, obinutuzumab, tositumomab, veltuzumab, GA101, TRU-015, PRO131921, or ibritumomab.

Other objects, features and advantages of the present invention will become apparent from the following. It should be understood, however, that the specific examples, while indicating preferred embodiments of the invention, are given by way of illustration only, since various changes and modifications within the spirit and scope of the invention will become apparent to those skilled in the art from this detailed description.

The following example(s) are given for the purpose of illustrating various embodiments of the invention and are not meant to limit the present invention in any fashion.

### Example 1

### Materials and Methods

### Induction of Active EAE

C57BL/6 6-8 week old females were actively immunized, maintained, handled and surveiled as outlined (29). Briefly, C57BL/6 6-8 week old females (Jackson Labs, Bar Harbor, Me.) were actively immunized by subcutaneous injection (s.c.) of 0.2 ml inoculum containing 200 μg of Myelin Oligodendrocyte Glycoprotein peptide fragment 35-55 (MOG peptide 35-55) (MEVGWYRSPFSRVVHLYRNGK; SEQ ID NO: 1) in IFA (DifcoLabs, Detroit, Mich.) with 800 μg Mycobacterium tuberculosus hominis H37Ra (MT) on day 0 and 7 following (30), with pertussis toxin (PTx) (List Biologicals) 200 ng i.p. on day 0 and day 2 and followed for evidence of disease. Severity was graded daily as follows by a blinded observer: 0=no disease; 1=minimal or mild hind limb weakness (associated with limp tail); 2=moderate hind limb weakness or mild ataxia (waddling gait and/or poor righting ability); 3=moderate to severe hind limb weakness; 4=severe hind limb weakness or moderate ataxia; 5=paraplegia with no more than moderate four limb weakness; 6=paraplegia with severe four limb weakness or severe ataxia.

### Adoptive Transfer

Thirty seven days after inoculation and after peak score of attack, all spleens from each treatment group were aseptically removed, single cell suspensions prepared, and red cell lysis performed by adding 2-3 ml sterile water to single cells for 5 seconds, and once the solution became transparent, adding AIM-V media to a 50 ml tube. Splenocytes from grouped IgG isotype control fed, 1 μg or 10 μg Rituxan fed mice were re-stimulated with MOG peptide 35-55 at a final concentration of 10 μg/ml for 48 hours in serum free medium (AIM-V medium, Gibco BRL, Grand Island, N.Y.) with 2×10⁵ cells/200 ml in triplicate in 96 well U-bottomed plates in a humidified 5% CO₂/95% air incubator at 37° C. Splenocytes from control fed mice were also re-stimulated with MOG peptide 35-55 and 50 μg/ml Rituxan in vitro as described above. CD4⁺ T cells were isolated from splenocytes after MOG restimulation above using CD4 (L3T4) MicroBeads (Miltenyi Biotec, Auburn, Calif.). Following incubation, cells were collected, washed twice in PBS, and viability determined by standard Trypan blue exclusion. Viable cells were adjusted to 10⁷ cells/0.5 ml Dulbecco's PBS immediately prior to i.p. injection into active MOG peptide 35-55 immunized recipient mice during ongoing disease (˜day 17 post immunization). Following administration of Rituxan or adoptive transfer, outcome was measured by comparing the difference between group mean active treatment and placebo group scores from day 17-32 post immunization.

### Active Protein

Rituximab (IgG₁) (RTX) (Rituxan®) was purchased from Roche Pharma.

### Control Protein

Mouse and human IgG₁ isotype control antibody (1-10 μg feeding, 50 μg/ml in vitro), was purchased from Southern Biotech, Birmingham, Ala.

### Dosing (Feeding) Regime

Once non-treated inoculated mice attained a score ˜2.0, B6 mice were randomized to one of 3 treatment groups, and gavaged (fed) with 0.1 ml of 1 μg or 10 μg mouse IgG₁ isotype control (mock), 1 μg, or 10 μg of Rituxan using a 2.5 cm syringe fitted with a 22-24 gauge ball point needle (Thomas Scientific, Swedesboro, N.J.) as described (31).

### Histology

Following sacrifice, cords were removed and immersion fixed in 10% neutral buffered formalin for a minimum of two weeks. After fixation, cords were sectioned in entirety in the horizontal plane at approximately 3 mm intervals and processed to paraffin. Paraffin blocks were sectioned at 6-8 microns, and step sections were stained with hematoxylin and eosin and examined by light microscopy. Cord sections were evaluated independently for foci of inflammation by an observer (SAB) (blinded) without knowledge of the treatment status of the mice prior to sacrifice. Spinal cord tissue was sampled in an identical fashion for each animal and numbers of inflammatory foci per high-powered field (HPF) (>20 perivascular lymphocytes) in the parenchyma were counted.

### Measurement of Cytokine Secretion

Spleens and spinal cords (CNS) from each treatment group were aseptically removed and single cell suspensions prepared. In spinal cords, whole cords were passed through a cell strainer for CNS lymphocytes (B and D, Franklin Lakes, N.J.) and spun at 600 rpm several times to separate lymphocytes from CNS tissue. Spleen leucocytes and cord lymphocytes from grouped IgG isotype control fed or 1 mg RTX fed mice were stimulated with 1 μg MOG peptide 35-55×48 hours as described (29). Murine cytokine responses were examined using a customized RayBio Mouse Cytokine Inflammatory Antibody Array that included innate cytokine TNF-α, IL-17 (Teff), Th1-like (IL-2, IFNγ), Th2-like cytokines (IL-4, IL-10, IL-13) and IL-12p70 using the RayBioantibody array Analysis tool application (RayBiotech, Inc, Norcross, Ga.). Mouse TGF-β was measured using Human/Mouse TGF-b1 ELISA Ready-SET-Go (eBioscience, San Diego, Calif.). Results were grouped from mice fed IgG isotype control or mice fed with RTX from grouped samples of at least two separate experiments (each sample performed in duplicate) and expressed as pg/ml ±SEM (student t-test).

### Phenotypic Analysis

CD25 and FoxP3 expression by CD3⁺CD4⁺ lymphocytes was analyzed using the Beckman Coulter 10-Color Gallios Flow Cytometer and mouse regulatory T Cell Staining Kit with PE FoxP3 FJK-16s, FITC CD4, APC CD25 (eBioscience, San Diego, Calif.) following the manufacturer's instructions. CD20 B cells were measured with anti-mouse CD20 antibodies (eBioscience, San Diego, Calif.) following the manufacturer's instructions.

### Statistics

Statistical analysis was performed using ANOVA and student t test (Prism 4.0).

### Example 2

### Results

Oral RTX Inhibits Active EAE and Donor Cells Transferred from RTX Fed Mice can Modulate Disease in Actively Immunized Recipients

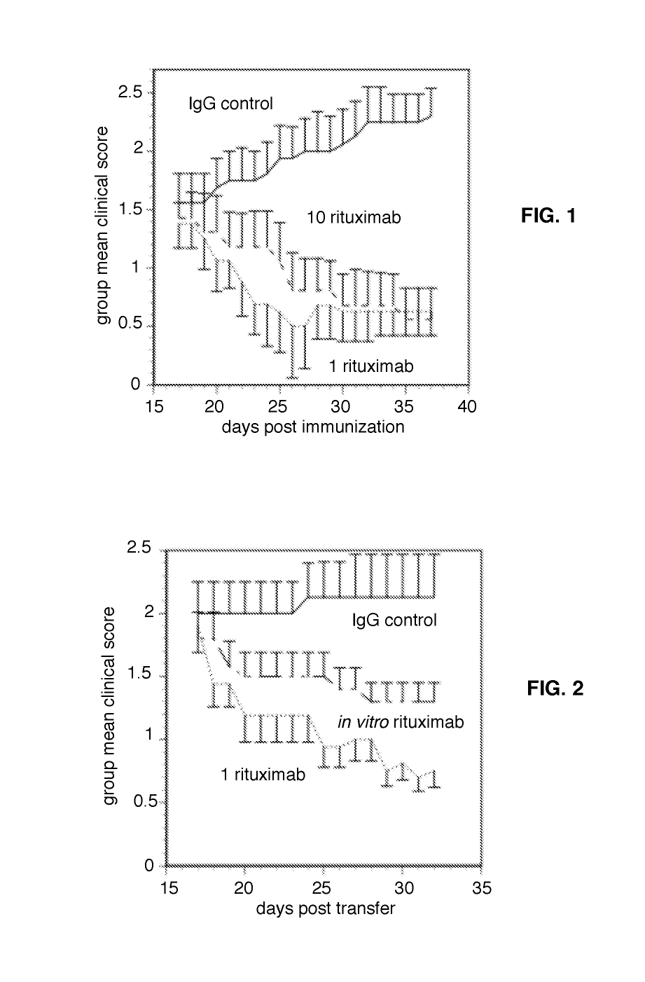

Preliminary experiments determined the immuno-modulatory capability of 1 mg and 10 mg ingested (orally administered) Rituxan compared to IgG control in EAE. Mice were immunized and separated into 3 groups once each mouse attained a score ˜1.5 (day 17 post immunization) at which time oral dosing was started. The IgG control group increased group score from day 17 and plateaued at score=2.3 37 days after inoculation and 20 days after the initiation of feeding. Active treatment groups fed with 1 and 10 mg showed significant decreases in group scores after initiation of therapy (day 17) with 1 mg showing the most effect and reduction of disease severity compared to placebo (FIG. 1).

Thirty seven days following immunization, there were significantly less inflammatory foci in the 1 mg fed group (mean group inflammatory score=7.3±1.2) compared to the IgG control fed group (mean group inflammatory score=21.5±4.0) (p<0.001, t test; n=24/group).

Whether passively transfer protection from Rituxan fed mice into actively immunized mice. After adoptive transfer of MOG-restimulated splenocytes into actively immunized recipient mice with early disease on day 17 (mean group score ˜1.8-2.0), recipients of donor splenocytes from IgG control fed mice increased their group disease severity over 23 days to a maximum of 2.2. In contrast, recipients of donor splenocytes from 1 mg Rituxan fed mice or from splenocytes incubated with 50 mg/ml Rituxan in vitro decreased their group score at day 32 to a score=0.7 and 1.3 respectively (FIG. 2). There was a significantly better score with 1 mg Rituxan fed group vs the 50 mg/ml Rituxan in vitro group.

Fifteen days following adoptive transfer, the number of CNS inflammatory foci in the IgG control fed group was significantly higher (mean group inflammatory score=24.6±4.5) compared to either 1 mg Rituxan fed donors (12.5±2.2) or in vitro Rituxan treated recipients (16.0±3.2) (p<0.008, ANOVA; n=12/group).

Adoptively Transferred CD4⁺ T Cells from RTX Fed Donor Mice can Modulate Disease in Actively Immunized Recipients

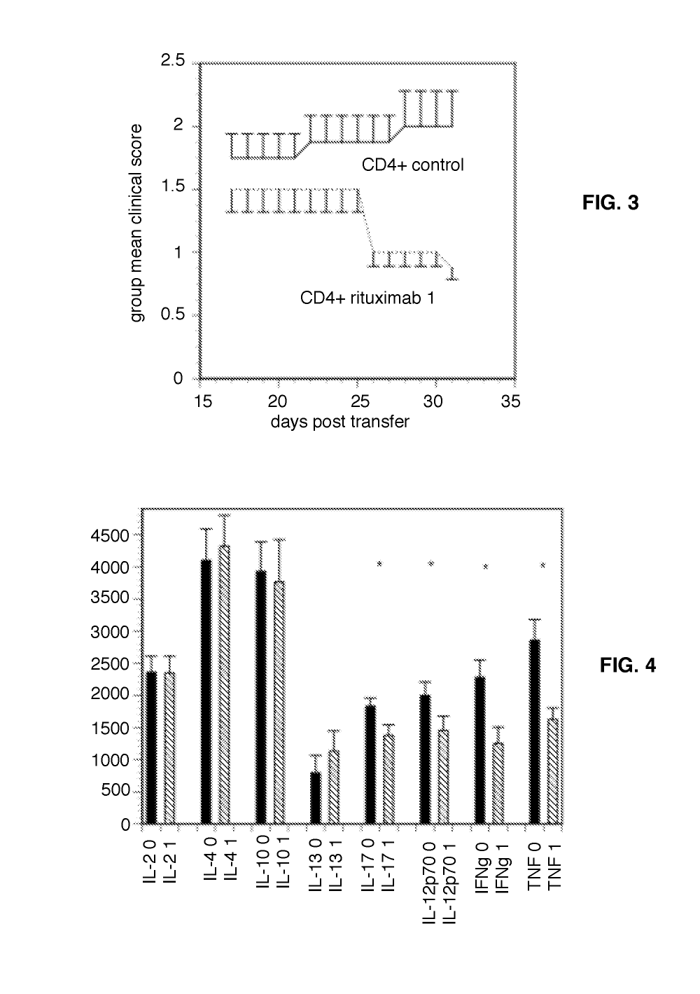

Whether a T cell subset (CD4⁺) from a fed donor would show immune-modulatory activity in actively immunized recipients was determined. After adoptive transfer of MOG-restimulated CD4⁺ T cells isolated from MOG activated splenocytes into actively immunized recipient mice with early disease on day 17 (mean group score ˜1.4-1.8), recipients of donor CD4⁺ T cells from IgG control fed mice increased their group disease severity over 14 days to a maximum of 2.0. In contrast, recipients of donor CD4⁺ T cells from 1 mg Rituxan fed mice decreased their group score at day 31 to a score=0.85 (FIG. 3).

Thirteen days following adoptive transfer, the number of CNS inflammatory foci in the IgG control fed group was significantly higher (mean group inflammatory score=20.6±7.4) compared to 1 mg Rituxan fed donors (6.3±2.0) (p<0.001, t test; n=12/group).

Oral RTX Decreases Pro-Inflammatory Cytokines in RTX Fed Mice and Recipients of Donor Cells from RTX Fed Mice

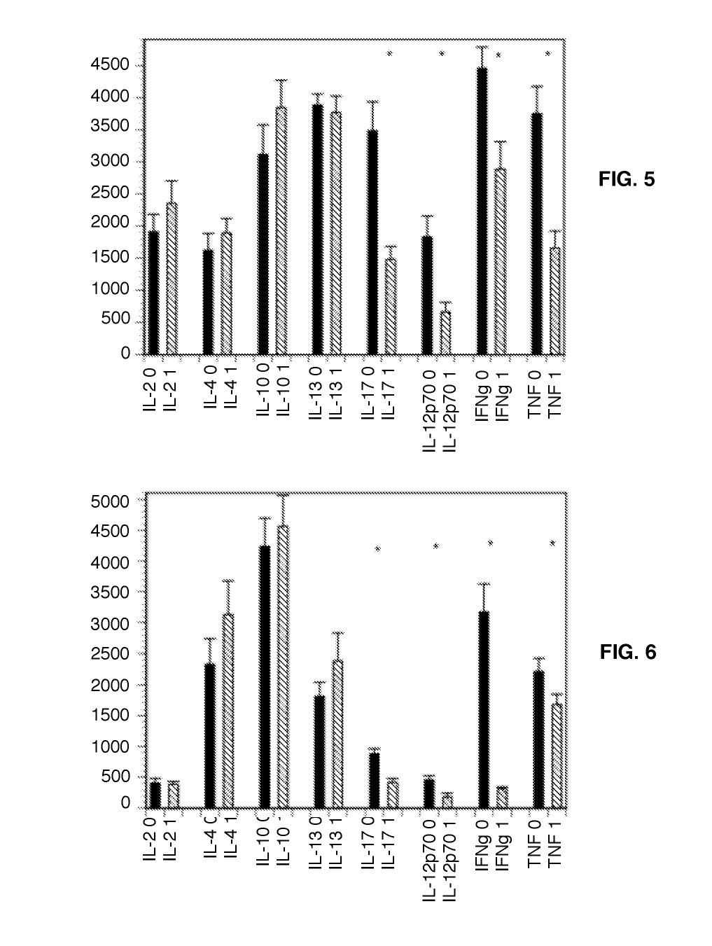

The effect of oral Rituxan on cytokines in actively fed and recipients of donor cells from fed mice was examined. The cytokine profiles of MOG re-stimulated spleen and cord lymphocytes in IgG fed control versus 1 mg Rituxan fed mice was compared. Splenic lymphocytes showed significant decreases in levels of Th1-like cytokines TNF-α, IL-12/IFN-γ and IL-17 in the Rituxan fed group compared to the IgG control fed group (FIG. 4). There was no significant change in peripheral splenic lymphocyte production of IL-4, IL-10 or IL-13 in Rituxan fed vs IgG control fed mice (FIG. 4). CNS lymphocytes showed significant decreases in levels of Th1-like cytokines TNF-α, IL-12/IFN-γ and IL-17 in the Rituxan fed group compared to the IgG control fed group (FIG. 5). There was no increase in CNS lymphocyte production of IL-4, IL-10 or IL-13 in Rituxan fed vs IgG control fed mice (FIG. 5).

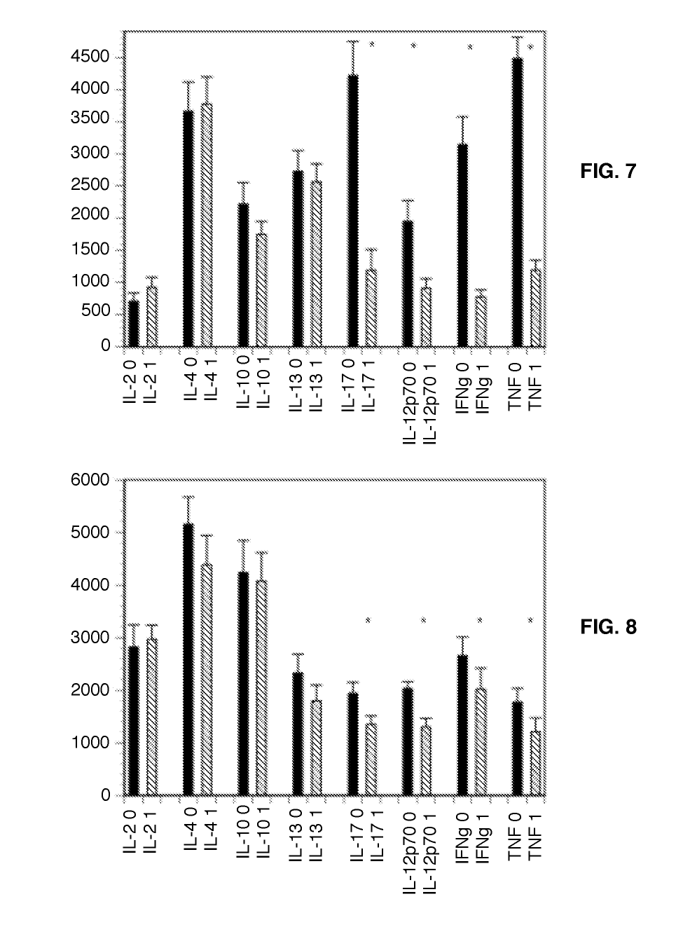

The cytokine profiles of MOG re-stimulated spleen and cord lymphocytes in recipients of IgG control fed vs Rituxan fed donor cells was examined. Splenic lymphocytes showed significant decrease in levels of IL-12, IL-17, IFN-γ and TNF-α in Rituxan fed groups compared to the IgG control fed group (FIG. 6). CNS lymphocytes showed significant decreases in levels of IL-12, IL-17, IFN-γ and TNF-α and no increases in counter-regulatory IL-4, IL-10 or IL-13 in the Rituxan fed group compared to the IgG control fed group (FIG. 7).

### Recipients of RTX Fed Spleen CD4⁺ T Cells Produce Less IL-12, IL-17, IFN-γ and TNF-α

Whether a CD4+ T cell subset lymphocyte showed differential activity on cytokine profiles was examined. The cytokine profiles of MOG re-stimulated spleen lymphocytes in recipients of IgG control fed CD4⁺ T cells vs Rituxan fed CD4⁺ T cells or donor cells was examined (from FIGS. 3-4). Recipient splenic lymphocytes showed significant decreased Th1-like cytokines TNF-α, IL-12/IFN-γ and IL-17 after CD4⁺ T cells transfer in Rituxan fed groups without significant changes in IL-4, IL-10 or IL-13 (FIG. 8). There were no significant changes in IL-2, IL-4, IL-10 or IL-13 in recipient spleen after CD4⁺ T cell from Rituxan fed donors.

Actively RTX Fed or Recipients of RTX Fed Cells Show No Significant Increase in CD4⁺CD25⁺FoxP3⁺ Cell, B Cells Frequency or TGF-β Secretion

Whether immunomodulation was due to increase T_{reg} frequency, decrease B cells or increased TGF-b secretion was also determined. Whether CD4⁺CD25⁺FoxP3⁺ T_{reg} might be induced by Rituxan feeding and therefore explain protection in actively treated and recipients of adoptively transferred cells from Rituxan fed donors was examined. FACS analysis shows no significant increase in CD4⁺CD25⁺FoxP3⁺ cell frequency or decreased B cell frequency in Rituxan fed compared to IgG control fed mice in actively fed or recipients of actively fed donor cells (data not shown).

Whether there was increased secretion of TGF-β in MOG re-stimulated spleen and cord lymphocytes from actively treated and recipients of adoptively transferred cells from Rituxan fed donors was examined. There was no effect on TGF-β secretion after active Rituxan feeding or in recipients of Rituxan feed donors in spleen or spinal lymphocytes compared to control (data not shown).

### DISCUSSION

The present invention shows an overall anti-inflammatory effect of ingested Rituxan in MOG immunized mice. Both 1 mg and 10 mg ingested (oral) Rituxan showed significant clinical effect with 1 mg demonstrating the most robust activity. Adoptive transfer of Rituxan fed or in vitro Rituxan treated MOG-re-stimulated splenocytes or CD4⁺ T cells into recipient mice with early disease suppressed ongoing disease. Both active treatment with oral Rituxan or adoptive transfer of splenocytes or CD4⁺ T cells from Rituxan fed or in vitro treated mice showed significantly less CNS inflammation in the Rituxan groups. There was a significant improvement in scores with 1 mg Rituxan fed donor splenocytes in recipients compared to the 50 mg/ml Rituxan in vitro group suggesting a unique effect from feeding.

Overall there was a decrease in innate inflammatory cytokine TNF-α, Th1-like cytokine IFN-γ, IL-12p70 and IL-17 in spleen and CNS without increases in Th2-like IL-4, IL-10, or IL-13 cytokines in active fed spinal cords and adoptive transfer recipient spinal cords.

B cells have paradoxical roles in autoimmunity exerting both pathogenic and protective effects. On the one hand, ablation with B cell-targeted mAb anti-CD20 indicate that B cells control the extent of EAE clinical disease (16). On the other hand, myelin-specific B cells and their antibodies play a role in EAE and MS (32). Spontaneous EAE Tg MOG 92-106 depends on the presence of an intact B cell compartment that produce pathogenic autoantibodies (23). B cells are required for the generation of CD4+ T cells specific for CNS auto-antigen and the entry of encephalitogenic T cells into the CNS during disease progression (28). B cells take up Ag through Ig receptors in vivo and prime naive CD4+ T cells both in vitro and in vivo showing that T cell priming can involve Ag-specific B cells (33).

Physiologically, the CD20 ligand enables optimal B-cell immune response specifically against T-independent antigens (34). However, the CD20 ligand functions in B cell proliferation and Ig production in conjunction with T cells. Antibodies against the B1 molecule (CD20) can inhibit B cell proliferation by activated T cells (35) and immunoglobulin secretion (36). CD20 blockade could therefore decrease proliferation and antibody production.

The reciprocal roles for B cells during EAE immune-pathogenesis depend on the timing of B cell involvement during the course of disease (28). These results follow Matsushita as opposed to others (21) and show that inactivation of B cells during EAE progression (˜14 days after inoculation) dramatically inhibits disease (28).

No significant depletion in B cells in the experiments were detected consistent with the inability of monomeric forms of Rituxan to induce apoptosis (15). It is known that conventional IgG monomer (IgG unit) such as Rituxan, as opposed to dimers that can undergo crosslinking, produce a homogeneous immune-fluorescent ring around a cell indicative of binding via CD20 (37). The gavaged monomeric IgG Rituxan presumably binds to CD20 in the gut (GALT) or after absorption blocks B cell APC function and Ab production resulting in inhibition of EAE.

B cells can produce inflammatory cytokines such as TNF-α, IL-12, IFN-γ, and IL-17. Inhibition of CD20 would be expected to block secretion of these inflammatory proteins. TNF-α is an important inflammatory cytokine in EAE. B cells secrete pro-inflammatory TNF-α amplifying the ongoing immune response (38) important in CNS pathology (39) and inducing EAE (40-41) as part of a functional ‘type 1 cytokine’ unit (42). Adoptive transfer of EAE with TNF-α producing cells causes CNS inflammation (43-44). The reduction of TNF activity reduces severity of EAE (45).

IL-12 is also produced in B cells. IL-12 producing B cells can be identified in mice with type 1 immune responses (46-47, stimulates myelin-reactive T cells that correlate with CNS-infiltrating and encephalitogenic properties (48) and drives antigen specific cells (49) in CNS (50) contributing to disease exacerbation (51). Antibody inhibition of endogenous IL-12 in vivo after transfer prevents paralysis (52).

B cells can induce IFN-γ secretion and produce IFN-γ. Memory B cells from RRMS patients stimulate CD4+ T cells and IFN-γ secretion in response to MBP and MOG (53). B cells primed by Th1 cells and antigens (B effector-Be-1) make cytokines associated with type 1 responses such as IFN-g (54).

IL-17 is not made exclusively by T cells but also by B cells. Cellular sources of IL-17 in rheumatoid arthritis include a significant number of non-T cells including CD3(−) CD19(+) B cells (55), B cells in parasitic infections (56) and constitutive expression of IL-17R in murine splenic B cells (57). The reduction of B cell functional ‘type 1 cytokine’ unit and IL-17 would be expected to ameliorate EAE.

The following references are cited herein.
1. Alvord et al., Ann NY Acad Sci. 1965, 122:333-45.
2. Feuer et al., J Neuroimmunol. 1985, 10:159-66.
3. Raine et al., J Neurol Sci. 1977, 31:355-67.
4. Wisnewski et al., Ann Neurol. 1977, 1:144-8.
5. Brod et al., Interferon Cytokine Res. 2002, 22:1153-66.
6. Brod et al., Autoimmunity. 2011, 44:437-43.
7. Brod et al., J Neuroimmunol. 2008, 193:106-12.
8. Brod et al., J Neuroimmunol. 2011, 232:131-5.
9. Brod et al., J Neuroimmunol. 1995, 58:61-9.
10. Brod et al., J Interferon Cytokine Res. 1995, 15:115-22.
11. Barr et al., J Exp Med. 2012, 209:1001-10.
12. Ray et al., J Immunol. 2012, 188:3188-98.
13. Idusogie et al., J Immunol. 2000, 164:4178-84.
14. Janas et al., Clin Exp Immunol. 2005, 139:439-46.
15. Ghetie et al., 1392-1397. 2001, 97:1392-8.
16. Ray et al., J Neuroimmunol. 2011, 230:1-9.
17. Bar-Or et al., Ann Neurol. 2008, 63:395-400.
18. Hauser et al., N Engl J Med. 2008, 358:676-88.
19. Stuve et al., Arch Neurol. 2009, 66:259-61.
20. Fillatreau et al., Nat Immunol. 2002, 3:944-50.
21. Weber et al., Ann Neurol. 2010, 68:369-83.
22. Willenborg et al., J Neuroimmunol. 1983, 5:99-109.
23. Pollinger et al., J Exp Med. 2009, 206:1303-16.
24. Matsushita et al., J Clin Invest. 2008, 118:3420-30.
25. Monson et al., PLoS One. 2011, 6:e17103.
26. Freireich et al., Cancer Chemother Rep. 1966, 50(4):219-244.
27. Watanabe et al., Risk Analysis. 1992, 12:301-10.
28. Mahmood et al., J Clin Pharmacol. 2003, 43(7):692-97.
29. Brod et al., J Neuroimmunol. 2007, 183:89-95.
30. Tompkins et al., J Immunol. 2002, 168:4173-83.
31. Brod et al., J Interferon Cytokine Res. 2008, 28:25-30.
32. Weber et al., Biochim Biophys Acta. 2011, 1812:239-45.
33. Constant et al., J Immunol. 1999, 162:5695-703.
34. Kuijpers et al., J Clin Invest. 2010, 120:214-22.
35. Tedder et al., J Immunol. 1985, 135:973-9.
36. Tedder et al., Eur J Immunol. 1986, 16:881-7.
37. Zhang N. Three antibody based immunotherapeutic modalities for malignancies. A dissertation, University of Southern California. 2006:132.
38. Duddy et al., J Immunol. 2004, 172:3422-7.
39. Renno et al., J Immunol. 1995, 154:944-53.
40. Issazadeh et al., J Neurosci Res. 1995, 40:579-90.
41. Okuda et al., J Interferon Cytokine Res. 1998, 18:415-21.
42. Qiu et al., Am J Pathol. 2001, 158:1503-15.
43. Held et al., J Autoimmun. 1993, 6:311-22.
44. Waldburger et al., Am J Pathol. 1996, 148:375-82.
45. Selmaj et al., Ann Neurol. 1991, 30:694-700.
46. Ganapamo et al., Eur J Immunol. 2001, 31:3460-8.
47. Harris et al. Nat Immunol. 2000, 1:475-82.
48. Bagaeva et al., J Neuroimmunol. 2003, 137:109-16.
49. Segal et al., J Exp Med. 1996, 184:771-5.
50. Issazadeh et al., J Neuroimmunol. 1996, 69:103-15.
51. Smith et al., Am J Path. 1997, 150:1909-17.
52. Leonard et al., J Exp Med. 1995, 181:381-6.
53. Harp et al., Eur J Immunol. 2010, 40:2942-56.
54. Lund F E. Curr Opin Immunol. 2008, 20:332-8.
55. Schlegel et al., PLoS One. 2013, 8:e82580.
56. Bermejo et al., Nat Immunol. 2013, 14:514-22.
57. Shibui et al., Cytokine. 2012, 59:108-14.

While the invention has been described with reference to certain embodiments, those skilled in the art will appreciate that modifications may be made without departing from the scope of the invention. All patents and publications cited in this specification are indicative of the level of those skilled in the art to which the invention pertains.

1. A method for treating or delaying the onset of multiple sclerosis in a human subject comprising orally administering to the subject an effective dose of an anti-CD20 antibody.

2. The method of claim 1, wherein the anti-CD20 antibody is administered in a fully humanized antibody.

3. The method of claim 1, wherein said antibody is rituximab, ocrelizumab, ofatumumab, obinutuzumab, tositumomab, veltuzumab, GA101, TRU-015, PRO131921, or ibritumomab.

4. (canceled)

5. The method of claim 3, wherein rituximab is administered in a dose from about 0.1 mg to about 50 mg.

6. The method of claim 5, wherein rituximab is administered in a solid or liquid form.

7. The method of claim 1, wherein said rituximab administration decreases levels of IL-17, IL-12, TNF-α and IFN-γ.

8. The method of claim 1, further comprising administering a compound selected from the group consisting of a SIRS peptide, α-MSH, ACTH and SST.

9. A method of decreasing IL-17, IL-12, TNF-α and IFN-γ in a human subject with multiple sclerosis comprising orally administering to the subject an effective dose of dose of an anti-CD20 antibody.

10. The method of claim 9, wherein the anti-CD20 antibody is administered in a fully humanized antibody.

11. The method of claim 10, wherein said antibody is rituximab, ocrelizumab, ofatumumab, obinutuzumab, tositumomab, veltuzumab, GA101, TRU-015, PRO131921, or ibritumomab.

12. (canceled)

13. The method of claim 11, wherein rituximab is administered in a dose from about 0.1 mg to about 50 mg.

14. The method of claim 13, wherein rituximab is administered in a solid or liquid form.

15. The method of claim 9, further comprising administering a compound selected from the group consisting of a SIRS peptide, α-MSH, ACTH and SST.
